(19) United States
(12) Patent Application Publication
(10) Pub. No.: US 2013/0296459 A1
(43) Pub. Date: Nov. 7, 2013

(54) MEDICAL ADHESIVE COMPOSITION

(71) Applicants: Moon-Hee Sung, Daejeon (KR); Hiroshi Uyama, Shiga (JP); Iwamoto Mie, Osaka (JP); Jae-Chul Choi, Daejeon (KR); Haryoung Poo, Daejeon (KR); Chul Joong Kim, Daejeon (KR)

(72) Inventors: Moon-Hee Sung, Daejeon (KR); Hiroshi Uyama, Shiga (JP); Iwamoto Mie, Osaka (JP); Jae-Chul Choi, Daejeon (KR); Haryoung Poo, Daejeon (KR); Chul Joong Kim, Daejeon (KR)

(73) Assignees: Bioleaders Corporation, Daejeon (KR); Korea Research Institute of Bioscience and Biotechnology, Daejeon (KR); THE INDUSTRY AND ACACDEMIC COOPERATION IN CHUNGNAM NATIONAL UNIVERSITY, Daejeon (KR); Kookmin University Industry - Academic Cooperation Foundation, Seoul (KR)

(21) Appl. No.: 13/884,912

(22) PCT Filed: Nov. 10, 2011
(86) PCT No.: PCT/KR2011/008565

§ 371 Date: Jul. 9, 2013

(30) Foreign Application Priority Data
Nov. 10, 2010 (KR) 10-2010-0111804

(51) Int. Cl. A61L 24/04; C08K 5/1545; C08K 5/053

(52) U.S. Cl. CPC A61L 24/046; C08K 5/053; C08K 5/1545
USPC 523/118; 524/386; 524/27

(57) ABSTRACT

The present invention provides a medical adhesive composition containing poly-gamma-glutamic acid or its salt; and sugar or sugar alcohol, which is edible, water-soluble, anionic and biodegradable. The invention also provides a thickener composition containing poly-gamma-glutamic acid, which can be used as a moisture-absorbing agent, a moisturizing agent and a raw material for cosmetic products.

### TECHNICAL FIELD

The present invention relates to a medical adhesive composition, and more particularly to a medical adhesive composition comprising poly-gamma-glutamic acid or its salt; and sugar or sugar alcohol.

### BACKGROUND ART

The term “medical instant adhesive” means, in a broad sense, medical supplies, including adhesive plasters, surgical adhesives and hemostatics, and in a narrow sense, adhesives that are used directly in medical fields, including dermatology, vascular surgery, gastroenterology and plastic surgery. Because the medical instant adhesive comes into contact with the skin, it should be biocompatible, should not be toxic and harmful to the body, should be biocompatible, and should have a hemostatic effect. In addition, it should show an instantaneous adhesive property even in the presence of moisture and should not interfere with the healing of the body.

Medical adhesive materials which are currently practically used include cyanoacrylates, fibrin glues, gelatin glues, and polyurethanes. Octyl cyanoacrylate which is a medical tissue adhesive (commercially available under the trade name “Dermabond” from Closure Medical Corp., USA) was approved for marketing by the EC in August, 1997 and approved for use by the US FDA in 1998. Ethicon, a subsidiary of Johnson & Johnson, has exclusively marketed this product in about 50 countries, including USA, Europe and Japan, and this product has been increasingly used worldwide for medical applications, including laceration healing, and the suture of incisions after plastic surgery and reconstructive surgery. In addition, there have been active studies on tissue adhesives, including 1,2-isopropylideneglyceryl 2-cyanoacrylates, alkyl 2-cyanoacryloyl glycolates, and methoxypropyl cyanoacrylates containing poly(trioxyethylene oxalate), in view of biocompatibility and biodegradability.

There have been studies focused on applying bioactive materials such as ligand peptides, which a proteinase substrate and a specific type of cell, to the adhesive material fibrin in order to impart functionality to the fibrin.

Cohesion Corp. (USA) developed a fibrin-collagen composite tissue adhesive (CoStasis™), a hemostatic product containing thrombin and bovine collagen. The surgical glue “Tissel” was approved for use for heart bypass surgery, colorectal surgery, trauma and the like by the US FDA in 1998, and other several products are waiting for approval by the FDA. In the year 1996, Green Cross Co., Ltd. (Korea) developed a fibrin glue (commercially available under the trade name “Greenplast”), a biological tissue adhesive that also serves as a hemostatic agent.

A local hemostatic agent, obtained by spinning alkali-solubilized collagen, making the collagen linear and adding a thermal crosslinking agent thereto, and a chitin-based hemostatic agent obtained by treating chitin with hydrochloric acid and processing the treated chitin to have a linear structure, have been developed. Recently, Collagenesis Corp. developed a photopolymerizable sealant using modified collagen. An albumin adhesive prepared by crosslinking albumin with modified polyethylene glycol has a low adhesive strength compared to fibrin, but it is expected that the shear strength thereof will increase with the passage of time so that the adhesive strength thereof exceeds that of fibrin glue. In the case of polyurethane-based adhesives, adhesives based on non-toxic fluorinated aliphatic diisocyanate having a slightly low curing rate, in place of aromatic diisocyanate which is toxic to the body, have been developed and clinically tested.

Poly-gamma-glutamic acid (γ-PGA) is a viscous material that is produced by microorganisms. Poly-gamma-glutamic acid is produced by Bacillus sp strains isolated from Chungkookjang (Korean traditional fermented soybean food produced using rice-straw), Natto (Japanese traditional fermented soybean food), Kinema (Nepali traditional fermented soybean food), etc. Poly-gamma-glutamic acid that produced by Bacillus sp. strains is an edible, water-soluble, anionic and biodegradable mer material that can be used as a moisture-absorbing agent, a moisturizing agent and a raw material for cosmetic products.

The present inventors obtained a patent relating to high-molecular-weight poly-gamma-glutamic acid and the use thereof (Korean Patent Registration No. 399091), and a patent relating to a method for producing poly-gamma-glutamic acid using a salt-tolerant Bacillus subtilis Chungkookjang strain that produces high-molecular-weight poly-gamma-glutamic acid (Korean Patent Registration No. 500796). In addition, the present inventors obtained patents relating to an anticancer composition, an immune adjuvant, an immune booster and an antiviral agent, which contain poly-gamma-glutamic acid (Korean Patent Registration Nos. 496606, 517114, 475406, and 873179). Further, the present inventors developed a hyaluronidase inhibitor containing poly-gamma-glutamic acid (Korean Patent Registration No. 582120) and found the immune boosting and anticancer functions of poly-gamma-glutamic acid (Poo, H. R. et al., Journal of Immunology, 178:775, 2007; Poo, H. R. et al., Cancer Immunol Immunother, 2009). Based on this finding, the present inventors have conducted studies on the various uses (including medical use) of poly-gamma-glutamic acid.

Accordingly, the present inventors have made extensive efforts to develop a medical adhesive using poly-gamma-glutamic acid, and as a result, have found that, when poly-gamma-glutamic acid is used in a mixture with sugar or sugar alcohol, it will show adhesive properties so that it can be used as a medical adhesive and a thickener for cosmetics, foods and the like, thereby completing the present invention.

### DISCLOSURE OF INVENTION

### Technical Problem

It is a main object of the present invention to provide a medical adhesive composition, which contains poly-gamma-glutamic acid or its salt and sugar or sugar alcohol and has adhesive properties.

### TECHNICAL SOLUTION

To achieve the above object, the present invention provides a medical adhesive composition containing poly-gamma-glutamic acid or its salt and sugar or sugar alcohol.

The present invention also provides a thickener composition containing poly-gamma-glutamic acid or its salt and sugar or sugar alcohol.

Other features and embodiments of the present invention will be more apparent from the following detailed descriptions and the appended claims.

### BRIEF DESCRIPTION OF THE DRAWINGS

FIG. 1 shows a mixed formulation of poly-gamma-glutamic acid and glycerol.

FIG. 2 shows a change in the viscosity of a mixed formulation of poly-gamma-glutamic acid and glycerol.

FIG. 3 shows a change in the viscosity of a mixed formulation of poly-gamma-glutamic acid and oligosaccharide.

FIG. 4 shows the change in the viscosity of a mixed formulation of poly-gamma-glutamic acid and oligosaccharide as a function of the concentration of oligosaccharide.

### BEST MODE FOR CARRYING OUT THE INVENTION

Unless defined otherwise, all technical and scientific terms used herein have the same meaning as commonly understood by one of ordinary skill in the art to which the invention pertains. Generally, the nomenclature used herein and the experiment methods which will be described later are those well known and commonly employed in the art.

The present invention is directed to a medical adhesive composition a thickener composition, which contain poly-gamma-glutamic acid or its salt and sugar or sugar alcohol.

The salt of poly-gamma-glutamic acid that is used in the present invention may be selected from the group consisting of sodium, potassium, calcium, ammonium, and zinc salts of poly-gamma-glutamic acid.

In the present invention, the poly-gamma-glutamic acid and its salt may have a molecular weight of 1-15,000 kDa.

The sugar that is used in the present invention may be selected from the group consisting of fructose, glucose, mannose, sucrose, maltose, trehalose, oligosaccharide, starch, and maltodextrin.

The sugar alcohol that is used in the present invention may be selected from the group consisting of xylitol, lactitol, isomalt, sorbitol, maltitol, and glycerol.

In the present invention, the medical adhesive composition may contain, based on 100 parts by weight of poly-gamma-glutamic acid, 0.05-5 parts by weight of the poly-gamma-glutamic acid salt, 1-99 parts by weight of sugar, and 1-99 parts by weight of sugar alcohol.

In the present invention, the thickener composition may contain, based on 100 parts by weight of poly-gamma-glutamic acid, 0.05-5 parts by weight of the poly-gamma-glutamic acid salt, 1-50 parts by weight of sugar, and 1-50 parts by weight of sugar alcohol.

In an example of the present invention, a poly-gamma-glutamic acid produced by Bicillus subtilis Chungkookjang. In addition, sodium, calcium and ammonium salts of the poly-gamma-glutamic acid were also used.

In another example of the present invention, in order to prepare a mixed formulation of the produced poly-gamma-glutamic acid and glycerol, a sodium salt of poly-gamma-glutamic acid was added to a glycerol solution, and the mixture was stirred until it had a strong viscosity so as not to flow down. Then, the viscosity of the mixture was measured, and as a result, it could be seen that the viscosity of the mixture increased as the molecular weight of the poly-gamma-glutamic acid added increased and as the concentration of the poly-gamma-glutamic acid added increased.

In another example of the present invention, in order to prepare a mixed formulation of the produced poly-gamma-glutamic acid and oligosaccharide, a sodium salt of the poly-gamma-glutamic acid was added to an oligosaccharide solution having a maltose content of 50%, followed by strong stirring. As a result, it could be seen that the viscosity of the mixture increased as the molecular weight of the poly-gamma-glutamic acid added increased and as the concentration of the poly-gamma-glutamic acid added increased. In addition, in order to observe the change in viscosity of the mixture as a function of the concentration of oligosaccharide, a sodium salt of the poly-gamma-glutamic acid was dissolved in oligosaccharide solutions having various oligosaccharide concentrations at a concentration of 1%, and the viscosities of the solutions were measured. As a result, it was shown that the viscosities increased as the oligosaccharide concentration increased, and particularly, a rapid increase in the viscosity appeared at an oligosaccharide concentration of 80%. This suggests that the mixture of poly-gamma-glutamic acid and oligosaccharide can show the highest viscosity when the mixing ratio between the poly-gamma-glutamic acid and the maltose contained in the oligosaccharide is suitable.

The inventive medical adhesive composition containing poly-gamma-glutamic acid or its salt and sugar or sugar alcohol may be used for an adhesive tape, a glue tape, a pre-taping foam underwrap, a pre-tape spray adhesive, adhesive dressing, a dressing gauze pad and the like, but is not limited thereto.

In the present invention, the medical adhesive composition preferably comprises, based on 100 parts by weight of poly-gamma-glutamic acid, 0.05-5 parts by weight of the poly-gamma-glutamic acid salt, 1-99 parts by weight of sugar and 1-99 parts by weight of sugar alcohol. More preferably, the medical adhesive composition preferably comprises, based on 100 parts by weight of poly-gamma-glutamic acid, 0.1-3 parts by weight of the poly-gamma-glutamic acid salt, 2-50 parts by weight of sugar and 2-50 parts by weight of sugar alcohol. If the contents of the poly-gamma-glutamic acid salt, sugar and sugar alcohol are out of the above ranges, the resulting composition will have insufficient adhesive properties, or increases in the contents will not lead to an increase in the adhesive effect of the composition. For example, if the content of the poly-gamma-glutamic acid salt is low and the content of sugar or sugar alcohol is lower than the lower limit of the above range, the adhesive property caused by the interaction between poly-gamma-glutamic acid and sugar or sugar alcohol will be reduced, and thus the composition cannot have an adhesive effect.

In the present invention, the thickener composition preferably comprises, based on 100 parts by weight of poly-gamma-glutamic acid, 0.05-5 parts by weight of the poly-gamma-glutamic acid salt, 1-50 parts by weight of sugar and 1-50 parts by weight of sugar alcohol. More preferably, the thickener composition preferably comprises, based on 100 parts by weight of poly-gamma-glutamic acid, 0.1-3 parts by weight of the poly-gamma-glutamic acid salt, 2-30 parts by weight of sugar and 2-30 parts by weight of sugar alcohol. If the contents of the poly-gamma-glutamic acid salt, sugar and sugar alcohol are out of the above ranges, the resulting composition will have insufficient adhesive properties, or increases in the contents will not lead to an increase in the adhesive effect of the composition. For example, if the content of the poly-gamma-glutamic acid salt is low and the content of sugar or sugar alcohol is lower than the lower limit of the above range, the increase in viscosity caused by the interaction between poly-gamma-glutamic acid and sugar or sugar alcohol will not appear, and thus the composition cannot have a thickening effect.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Preparation of High-Molecular-Weight Poly-Gamma-Glutamic Acid and its Salt

A poly-gamma-glutamic acid-producing basal medium (supplemented with 3% L-glutamic acid; 3% glucose, 1% (NH₄)₂SO₄, 0.27% KH₂PO₄, 0.17% Na₂HPO₄/12H₂O, 0.1% NaCl, 0.5% sodium citrate, 0.02% soypeptone, 0.7% MgSO₄/7H₂O, 10 Ml/l vitamin solution, pH 6.8) was prepared and sterilized, and a culture broth (LB medium) of Bacillus subtilis var Chungkookjang (KCTC 0697BP) was inoculated into the medium at a concentration of 4% and fermented in a 5 l jar fermentor (working volume: 3 l) at a stirring speed of 500 rpm, an air injection rate of 1.0 vvm and a temperature of 37° C. for 48 hours. Then, the microbial cells were removed through a filter press (containing diatomaceous earth), thereby obtaining a solution containing poly-gamma-glutamic acid. The solution containing poly-gamma-glutamic acid was adjusted to a pH of 2.0 using 2N sulfuric acid solution, and then allowed to stand at 10° C. for 15 hours, thereby obtaining a poly-gamma-glutamic acid precipitate. The obtained poly-gamma-glutamic acid precipitate was washed with a sufficient amount of cold distilled water (pH of 3.5 or higher) having a temperature of 10° C. or below, and was then passed through a Nutsche filter to collect the poly-gamma-glutamic acid. The collected poly-gamma-glutamic acid was freeze-dried, thereby obtaining a high-molecular-weight poly-gamma-glutamic acid.

In order to prepare salts of the poly-gamma-glutamic acid, to the poly-gamma-glutamic acid precipitate obtained by the precipitation process using sulfuric acid solution, the food additive sodium hydroxide was added to obtain a poly-gamma-glutamic acid sodium salt solution and a poly-gamma-glutamic acid calcium salt solution, which had a neutral pH. The salt solutions were freeze-dried to obtain poly-gamma-glutamic acid sodium salt powder and poly-gamma-glutamic acid calcium salt powder. In addition, the food additive ammonium bicarbonate was added to the poly-gamma-glutamic acid precipitate to obtain a poly-gamma-glutamic acid ammonium salt solution having a neutral pH. The ammonium salt solution was freeze-dried to obtain poly-gamma-glutamic acid ammonium salt powder.

### Example 2

### Preparation and Properties of Mixed Formulation of Poly-Gamma-Glutamic Acid and Glycerol

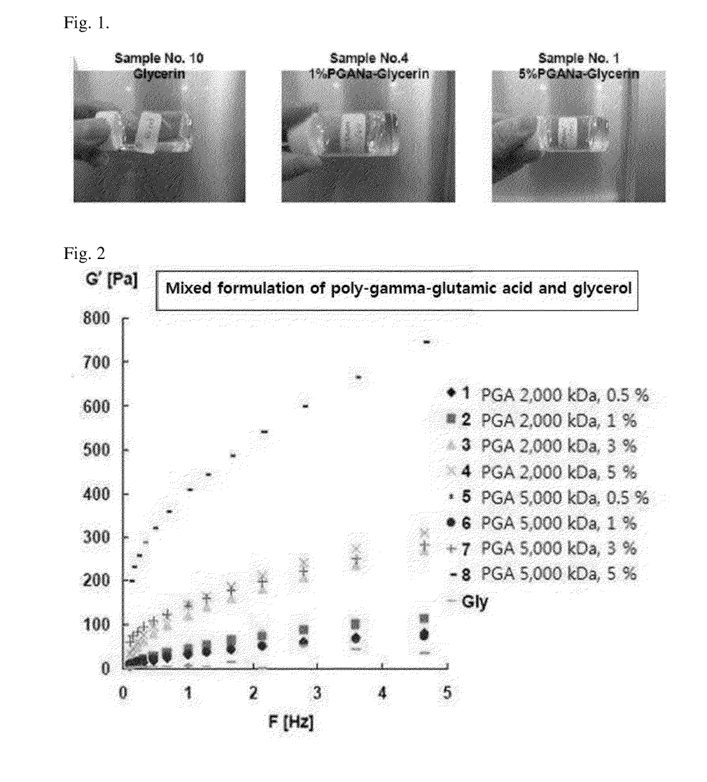

In order to prepare a mixed formulation of the poly-gamma-glutamic acid salt prepared in Example 1 and glycerol, each of the poly-gamma-glutamic acid sodium salts having a molecular weight of 2,000 kDa and 5,000 kDa was added to a glycerol solution at concentrations of 0.5%, 1%, 3% and 5% and was strongly stirred in an agitator (Poonglim, Korea). As a result, it was shown that the stirred mixtures showed a strong viscosity so as not to flow down. The viscosities of the mixtures were measured, and as a result, it could be seen that the viscosities increased as the molecular weight and concentration of the poly-gamma-glutamic acid added increased (see FIG. 2).

### Example 3

### Preparation and Properties of Mixed Formulation of Poly-Gamma-Glutamic Acid and Oligosaccharide

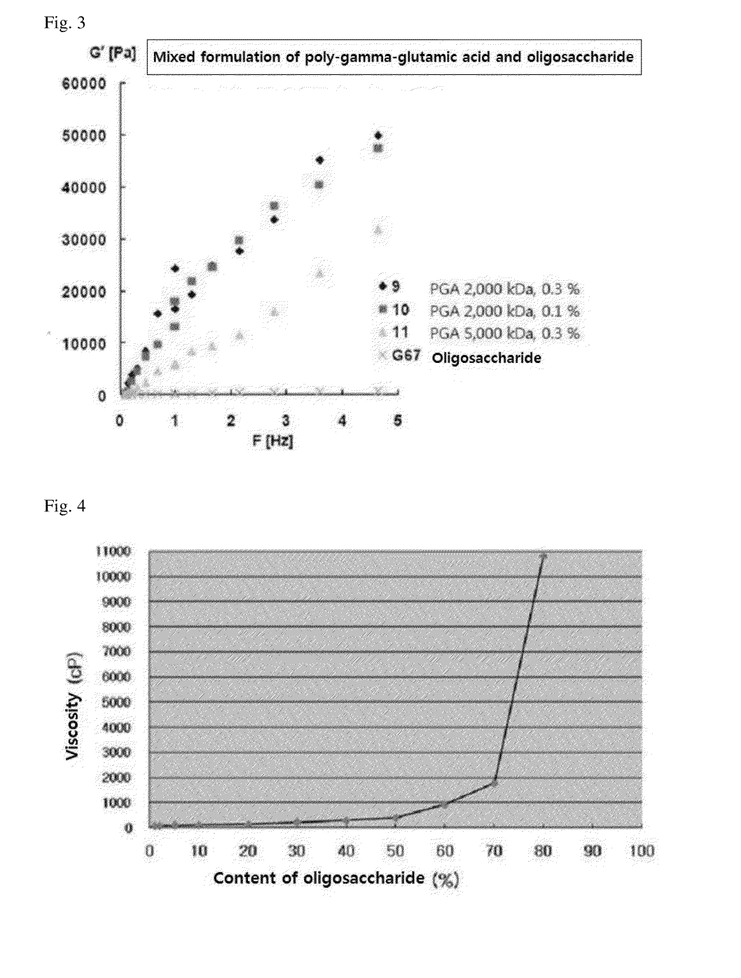

In order to prepare a mixed formulation of the poly-gamma-glutamic acid salt prepared in Example 1 and oligosaccharide, each of the poly-gamma-glutamic acid sodium salts having a molecular weight of 2,000 kDa and 5,000 kDa was added to an oligosaccharide solution having a maltose content of 50% at concentrations of 0.5%, 1%, 3% and 5% and was strongly stirred. As a result, as can be seen in FIG. 3, the viscosities of the mixtures increased as the molecular weight and concentration of the poly-gamma-glutamic acid added increased. In addition, in order to observe the change in viscosity of the mixture as a function of the oligosaccharide concentration, the poly-gamma-glutamic acid sodium salt was dissolved in oligosaccharide solutions having various oligosaccharide concentrations at a concentration of 1%, and the viscosities of the solutions were measured. As a result, it was shown that the viscosity increased as the oligosaccharide concentration increased, and particularly, a rapid increase in the viscosity appeared at an oligosaccharide concentration of 80% (see FIG. 4). This suggests that the mixture of poly-gamma-glutamic acid and oligosaccharide can show the highest viscosity when the mixing ratio between the poly-gamma-glutamic acid and the maltose contained in the oligosaccharide is 1:4.

### INDUSTRIAL APPLICABILITY

As described above, a medical adhesive composition containing poly-gamma-glutamic acid according to the present invention has edible, water-soluble, anionic and biodegradable properties. In addition, a thickener composition containing poly-gamma-glutamic acid according to the invention can be used as a moisture-absorbing agent, a moisturizing agent and a raw material for cosmetic products.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. A medical adhesive composition containing poly-gamma-glutamic acid or its salt and sugar or sugar alcohol.

2. The composition of claim 1, wherein the salt of poly-gamma-glutamic acid is selected from the group consisting of sodium, potassium, calcium, ammonium, and zinc salts of poly-gamma-glutamic acid.

3. The composition of claim 1, wherein the poly-gamma-glutamic acid and its salt have a molecular weight of 1-15,000 kDa.

4. The composition of claim 1, wherein the sugar is selected from the group consisting of fructose, glucose, mannose, sucrose, maltose, trehalose, oligosaccharide, starch, and maltodextrin.

5. The composition of claim 1, wherein the medical adhesive composition contains, based on 100 parts by weight of poly-gamma-glutamic acid, 0.05-5 parts by weight of the poly-gamma-glutamic acid salt, 1-99 parts by weight of sugar, and 1-99 parts by weight of sugar alcohol.

6. The composition of claim 1, wherein the sugar alcohol that is selected from the group consisting of xylitol, lactitol, isomalt, sorbitol, maltitol, and glycerol.

7. A thickener composition containing poly-gamma-glutamic acid or its salt and sugar or sugar alcohol.

8. The composition of claim 7, wherein the salt of poly-gamma-glutamic acid is selected from the group consisting of sodium, potassium, calcium, ammonium, and zinc salts of poly-gamma-glutamic acid.

9. The composition of claim 7, wherein the poly-gamma-glutamic acid and its salt have a molecular weight of 1-15,000 kDa.

10. The composition of claim 7, wherein the thickener composition contains, based on 100 parts by weight of poly-gamma-glutamic acid, 0.05-5 parts by weight of the poly-gamma-glutamic acid salt, 1-50 parts by weight of sugar, and 1-50 parts by weight of sugar alcohol.

11. The composition of claim 7, wherein the sugar that is selected from the group consisting of fructose, glucose, mannose, sucrose, maltose, trehalose, oligosaccharide, starch, and maltodextrin.

12. The composition of claim 7, wherein the sugar alcohol is selected from the group consisting of xylitol, lactitol, isomalt, sorbitol, maltitol, and glycerol.
